(19) United States
(12) Patent Application Publication
(10) Pub. No.: US 2025/0040931 A1
(43) Pub. Date: Feb. 6, 2025

(54) SURGICAL MEDICAL DEVICES

(71) Applicant: Covidien LP, Mansfield, MA (US)

(72) Inventors: Mingfei Chen, Santa Rosa, CA (US); Rhonda L. Staneart, Santa Rosa, CA (US); Emily E. Jacobs, Durham, CT (US); Gerald N. Hodgkinson, Killingworth, CT (US); Brian C. Graham, Santa Rosa, CA (US); Monideepa Chatterjee, Hamden, CT (US)

(73) Assignee: Covidien LP, Mansfield, MA (US)

(21) Appl. No.: 18/799,563

(22) Filed: Aug. 9, 2024

Related U.S. Application Data
(63) Continuation of application No. 18/113,811, filed on Feb. 24, 2023, now Pat. No. 12,070,213.
(60) Provisional application No. 63/313,302, filed on Feb. 24, 2022.

(51) Int. Cl. A61B 17/072

(52) U.S. Cl. CPC A61B 17/07292; A61B 2017/07257; A61B 2017/07271

(57) ABSTRACT

Surgical stapling devices have a surgical buttress attached to an anvil jaw member, a cartridge jaw member, or both. In use, the surgical buttress reinforces a staple line formed upon application of staples from the surgical stapling device and minimizes leaks which may exude from the staple line and/or tissue being stapled by the surgical stapling device.

### CROSS-REFERENCE TO RELATED APPLICATIONS

This application is a continuation of U.S. patent application Ser. No. 18/113,811 filed Feb. 24, 2023, which claims the benefit of the filing date of provisional U.S. Patent Application No. 63/313,302 filed on Feb. 24, 2022, and which applications are incorporated herein by reference in their entireties. To the extent appropriate a claim of priority is made to each of the disclosed applications.

### TECHNICAL FIELD

The present disclosure relates to surgical devices, such as surgical buttresses, for use with wound closure devices.

### BACKGROUND

Surgical stapling instruments are employed by surgeons to sequentially or simultaneously apply one or more rows of fasteners, e.g., staples or two-part fasteners, to body tissue for the purpose of joining segments of body tissue together. Such instruments generally include a pair of jaws or finger-like structures between which the body tissue to be joined is placed. When the stapling instrument is actuated, or “fired,” longitudinally moving firing bars contact staple drive members in one of the jaws. The staple drive members push the surgical staples through the body tissue and into an anvil in the opposite jaw, which forms the staples. If tissue is to be removed or separated, a knife blade can be provided in the jaws of the device to cut the tissue between the lines of staples.

When operating on certain tissue, such as lung, esophageal, intestinal, duodenal, and vascular tissue, it is important to effectively seal the tissue which can be particularly prone to air or fluid leakage. Preventing or reducing air or fluid leakage can significantly decrease post operative recovery time.

Improved surgical repair materials, capable of use as surgical buttresses for sealing and/or reinforcing staple lines in tissue, remain desirable.

### SUMMARY

The present disclosure relates to medical devices, including surgical stapling devices, which can be used to repair tissue.

In aspects, a surgical stapling device of the present disclosure includes an end effector having an anvil jaw member and a cartridge jaw member pivotally coupled to one another, the anvil jaw member and the cartridge jaw member being relatively movable such that the end effector is movable between an open position and a clamped position. A surgical buttress is attached to the anvil jaw member, the cartridge jaw member, or both.

In some aspects, a surgical stapling device of the present disclosure includes an end effector including an anvil jaw member and a staple cartridge jaw member pivotally coupled to one another, the anvil jaw member and the staple cartridge jaw member being relatively movable such that the end effector is movable between an open position and a clamped position, A surgical buttress may be attached to the anvil jaw member, the staple cartridge jaw member, or both, the surgical buttress including a substrate and a porous layer.

In some aspects, in use, the substrate of the surgical buttress is adjacent tissue, and the porous and/or swellable layer of the surgical buttress is adjacent the substrate of the surgical buttress.

In other aspects, in use, the porous and/or swellable layer of the surgical buttress is adjacent tissue, and the substrate of the surgical buttress is adjacent the porous and/or swellable layer of the surgical buttress.

In yet other aspects, in use, a first porous and/or swellable layer of the surgical buttress is adjacent tissue, the substrate of the surgical buttress is adjacent the first porous and/or swellable layer of the surgical buttress, and a second porous and/or swellable layer of the surgical buttress is adjacent the substrate of the surgical buttress.

In aspects, the surgical buttress further comprises a non-permeable layer.

In some aspects, in use, the substrate of the surgical buttress is adjacent tissue, the porous and/or swellable layer of the surgical buttress is adjacent the substrate of the surgical buttress, and the non-permeable layer of the surgical buttress is adjacent the porous and/or swellable layer of the surgical buttress.

In other aspects, in use, the porous and/or swellable layer of the surgical buttress is adjacent tissue, the substrate of the surgical buttress is adjacent the porous and/or swellable layer of the surgical buttress, and the non-permeable layer of the surgical buttress is adjacent the substrate of the surgical buttress.

In aspects, the substrate of the surgical buttress has a pore volume from about 65% to about 90%, and a thickness from about 0.05 mm to about 0.5 mm, in embodiments from about 0.1 mm to about 0.2 mm.

In some aspects, the porous and/or swellable layer of the surgical buttress has a pore volume from about 65% to about 99% and a non-compressed thickness from about 100 μm to about 2000 μm.

In other aspects, the non-permeable layer of the surgical buttress has a thickness from about 5 μm to about 50 μm.

In other aspects, a surgical stapling device of the present disclosure includes an end effector including an anvil jaw member and a staple cartridge jaw member pivotally coupled to one another, the anvil jaw member and the staple cartridge jaw member being relatively movable such that the end effector is movable between an open position and a clamped position. The surgical stapling device also includes a surgical buttress attached to the anvil jaw member, the staple cartridge jaw member, or both, the surgical buttress including a substrate, a porous layer, and a non-permeable layer.

In aspects, the present disclosure also provides a method for treating tissue, which includes stapling tissue with the recited surgical stapling device.

In aspects, a surgical buttress of the present disclosure includes a substrate having a pore volume from about 65% to about 85%, and a thickness from about 0.05 mm to about 0.5 mm; a porous and/or swellable layer having a pore volume from about 65% to about 99%, and a non-compressed thickness from about 100 μm to about 2000 μm; and a non-permeable layer having a thickness from about 5 μm to about 50 μm.

In some aspects, in use, the substrate of the surgical buttress is adjacent tissue, the porous and/or swellable layer of the substrate is adjacent the substrate of the surgical buttress, and the non-permeable layer of the surgical buttress is adjacent the porous and/or swellable layer of the surgical buttress.

### BRIEF DESCRIPTION OF THE DRAWINGS

Aspects of the presently disclosed surgical stapling apparatus are described herein with reference to the drawings wherein:

FIG. 1 is a perspective view of a surgical stapling apparatus including a handle housing, an adapter assembly, an end effector, and surgical buttresses attached to components thereof in accordance with an aspect of the present disclosure;

FIG. 2 is an enlarged view of the indicated area of detail shown in FIG. 1;

FIG. 3 is a perspective view of an anvil assembly of the end effector of the surgical stapling apparatus shown in FIG. 1, showing how a surgical buttress in accordance with an aspect of the present disclosure may be attached thereto;

FIG. 4 is a perspective view of a cartridge assembly of the end effector of the surgical stapling apparatus shown in FIG. 1, showing how a surgical buttress in accordance with an aspect of the present disclosure may be attached thereto;

FIG. 5 is a cross-sectional view taken along section line 5-5 of FIG. 2, showing the configuration of the surgical buttresses of the present disclosure attached to the anvil assembly and the cartridge assembly, prior to firing of the surgical stapling apparatus of FIG. 1;

FIG. 6 is a side view of a surgical buttress of the present disclosure;

FIG. 7 is a side view of an alternate surgical buttress of the present disclosure;

FIG. 8 is a side view of an alternate surgical buttress of the present disclosure;

FIG. 9 is a side view of an alternate surgical buttress of the present disclosure;

FIG. 10 is a side view of an alternate surgical buttress of the present disclosure; and

FIG. 11 is a scanning electron microscope image of a surgical buttress produced in accordance with Example 1 of the present disclosure.

### DETAILED DESCRIPTION

Various exemplary aspects of the present disclosure are discussed herein below in terms of surgical buttresses for use with tissue fixation devices, in aspects surgical staples. While the below disclosure discusses in detail the use of these surgical buttresses with staples, it will be appreciated that surgical stapling apparatuses of the present disclosure include a range of surgical buttressing materials and film-based materials that may be used to mechanically support tissues, reinforce tissues along staple or suture lines, and decrease the incidence of fluid leakage and/or bleeding of tissues. For example, other suitable materials which may be used with the surgical stapling apparatus of the present disclosure include hernia patches and/or tissue scaffolds.

The surgical buttress used with a surgical stapling apparatus of the present disclosure is in the form of a generally rectangular body having a distal end and a proximal end, with opposing lateral sides that run along the length of the elongate rectangular body portion from the distal end to the proximal end.

In aspects, a surgical buttress of the present disclosure may be formed of multiple layers. The layers may include a substrate, a porous and/or swellable layer, a non-permeable layer, and any combination thereof. In some aspects, the substrate may be a non-woven substrate. As used herein, a “porous layer” encompasses a layer that is porous and/or swellable, as further disclosed and/or described herein.

It should be understood that a variety of surgical stapling apparatuses may be utilized with a surgical buttress of the present disclosure. In aspects, linear staplers may be utilized such as, for example, those including EndoGIA™ Reinforced Reload with Tri-Staple Technology™ and other staplers with Tri-Staple™ technology, available through Medtronic, (North Haven, CT), as well as other anastomosis staplers, such as, for example, EEA™, CEEA™, GIA™, EndoGIA™, and TA™, also available through Medtronic. It should also be appreciated that the principles of the present disclosure are equally applicable to surgical staplers having alternate configurations, such as, for example, end-to-end anastomosis staplers having a circular cartridge and anvil (see, e.g., commonly owned U.S. Pat. No. 5,915,616, entitled “Surgical Fastener Applying Apparatus,” the entire disclosure of which is incorporated by reference herein); laparoscopic staplers (see, e.g., commonly owned U.S. Pat. Nos. 6,330,965 and 6,241,139, each entitled “Surgical Stapling Apparatus,” the entire disclosures of each of which are incorporated by reference herein); and transverse anastomosis staplers (see, e.g., commonly owned U.S. Pat. Nos. 5,964,394 and 7,334,717, each entitled “Surgical Fastener Applying Apparatus”, the entire disclosures of each of which are incorporated by reference herein).

Aspects of the presently disclosed surgical buttress and surgical stapling apparatus will now be described in detail with reference to the figures wherein like reference numerals identify similar or identical elements. In the following discussion, the terms “proximal” and “trailing” may be employed interchangeably, and should be understood as referring to the portion of a structure that is closer to a clinician during proper use. The terms “distal” and “leading” may also be employed interchangeably, and should be understood as referring to the portion of a structure that is further from the clinician during proper use. As used herein, the term “patient” should be understood as referring to a human subject or other animal, and the term “clinician” should be understood as referring to a doctor, nurse, or other care provider and may include support personnel.

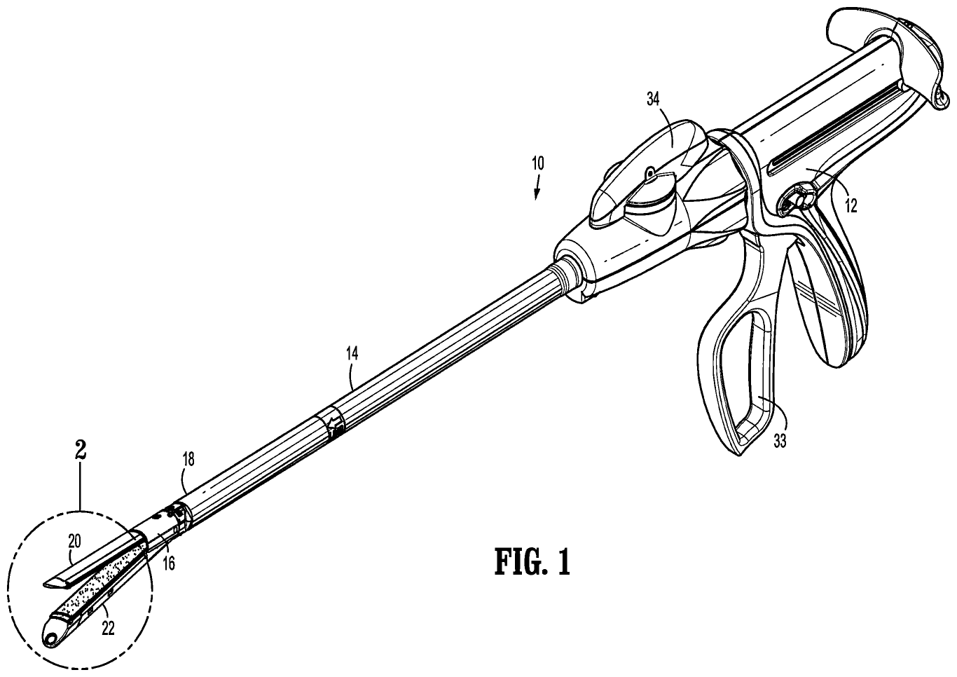

Referring now to FIG. 1, there is disclosed an exemplary surgical stapling apparatus or surgical stapler 10 for use in stapling tissue and applying a surgical buttress to tissue. The surgical stapling apparatus 10 generally includes a handle 12 having an clongate tubular member 14 extending distally from the handle 12. An end effector 16 is mounted on a distal end 18 of the elongate tubular member 14. The end effector 16 includes an anvil assembly including a staple clinching anvil jaw member 20 and a cartridge assembly including a staple cartridge jaw member 22 configured to receive a staple cartridge 32. The end effector 16 may be permanently affixed to the elongate tubular member 14 or may be detachable and thus replaceable with a new end effector 16. The staple clinching anvil jaw member 20 is movably mounted on the distal end 18 of the end effector 16 and is movable between an open position spaced apart from the staple cartridge jaw member 22 to a closed position substantially adjacent the staple cartridge jaw member 22.

The surgical stapling apparatus 10 further includes a trigger 33, as seen in FIG. 1, movably mounted on the handle 12. Actuation of the trigger 33 initially operates to move the anvil jaw member 20 from the open to the closed position relative to the staple cartridge jaw member 22 and subsequently actuates the surgical stapling apparatus 10 to apply lines of staples to tissue. In order to properly orient the end effector 16 relative to the tissue to be stapled, the surgical stapling apparatus 10 is additionally provided with a rotation knob 34 mounted on the handle 12. Rotation of the rotation knob 34 relative to the handle 12 rotates the elongate tubular member 14 and the end effector 16 relative to the handle 12 so as to properly orient the end effector 16 relative to the tissue to be stapled.

Reference may be made to commonly owned U.S. Pat. Nos. 5,915,616, 6,330,965, and 6,241,139, the disclosures of each of which are incorporated by reference herein, for a detailed discussion of the construction and operation of the surgical stapling apparatus 10.

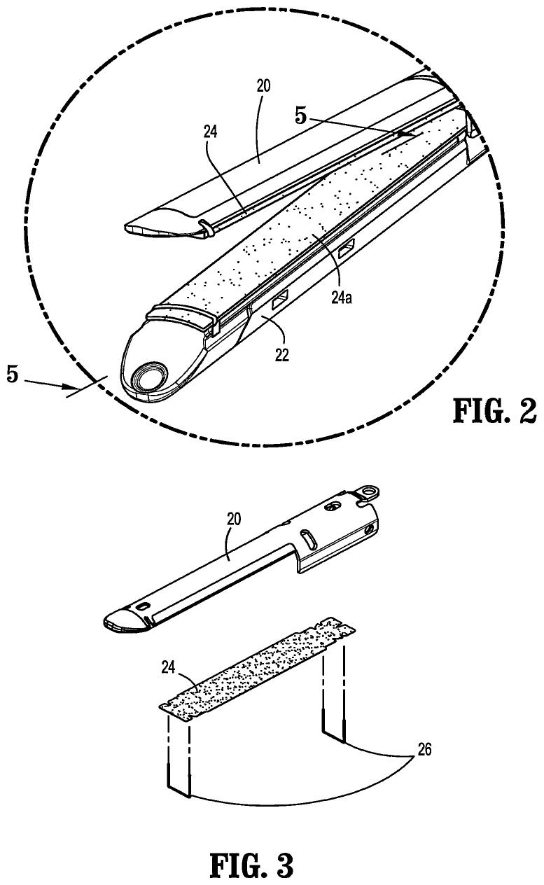

Referring to FIGS. 2-4, the staple clinching anvil jaw member 20 and the staple cartridge jaw member 22 may be provided with surgical buttresses 24, 24a. The surgical buttresses 24, 24a are provided to reinforce and seal staple lines applied to tissue by the surgical stapling apparatus 10. The surgical buttresses 24, 24a may be configured in any shape, size, or dimension suitable to fit any surgical stapling, fastening, or firing apparatus.

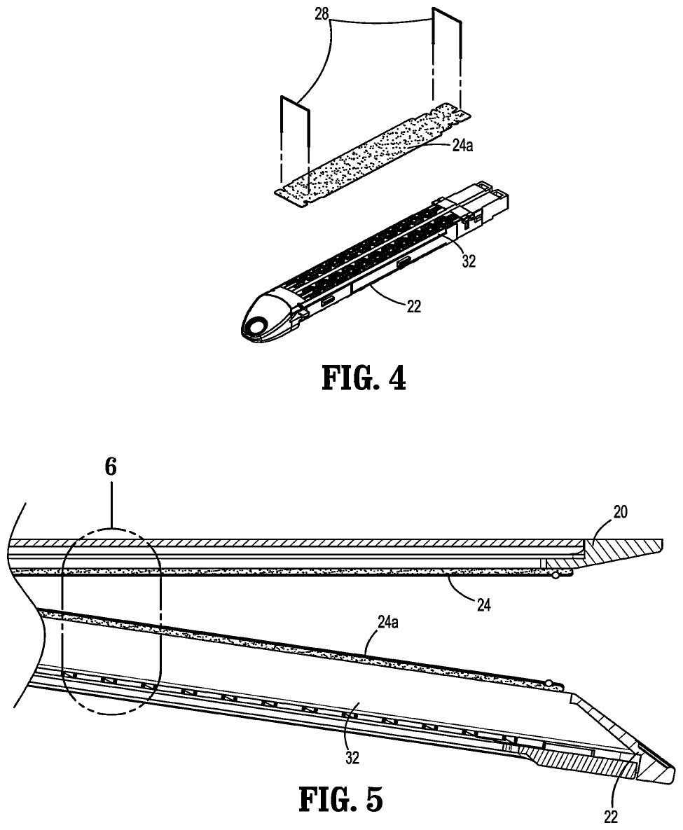

As illustrated in FIG. 3, the surgical buttress 24 may be attached to the staple clinching anvil jaw member 20 with sutures 26. Similarly, as illustrated in FIG. 4, the surgical buttress 24a may be attached to the staple cartridge jaw member 22 with sutures 28.

As illustrated in FIGS. 3-5, the staple clinching anvil jaw member 20 has been loaded with a surgical buttress 24, and the staple cartridge jaw member 22, including the staple cartridge 32, has been loaded with a surgical buttress 24a.

Although not depicted in the Figures, in some aspects a surgical stapler 10 of the present disclosure may have the surgical buttress 24 on the staple clinching anvil jaw member 20, the surgical buttress 24a on the staple cartridge jaw member 22, but not both.

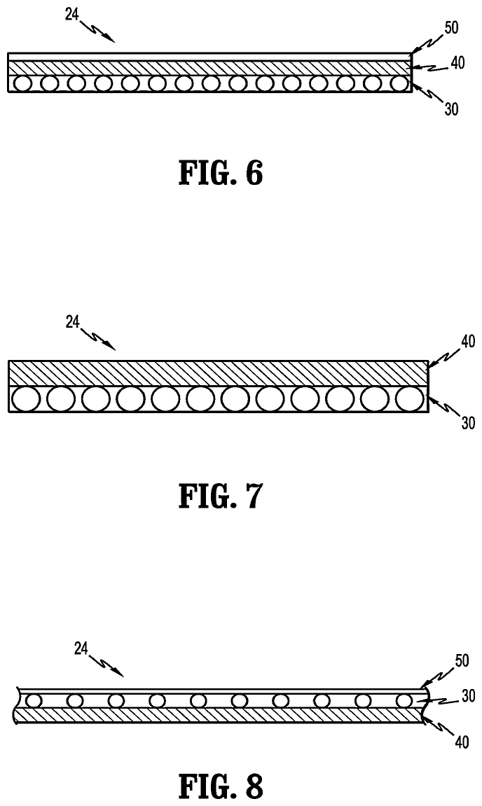

With general reference to FIGS. 6 and 8, the surgical buttress 24 of the present disclosure may include multiple layers. The surgical buttress 24, for example, may include a substrate 30, a porous and/or swellable layer 40 and a non-permeable layer 50.

Any porous and/or swellable portion of a surgical buttress of the present disclosure, including the substrate 30 and/or the porous and/or swellable layer 40, may have openings or pores over at least a part of a surface thereof. Suitable porous and/or swellable materials for forming these layers include, but are not limited to, fibrous structures (e.g., knitted structures, woven structures, non-woven structures, etc.) and/or open cell foams.

Porous and/or swellable portions of the surgical buttress of the present disclosure may include fibrous materials, formed using any suitable method including, but not limited to, knitting, weaving, non-woven techniques (including melt blowing), wet-spinning, electro-spinning, extrusion, co-extrusion, and the like. Examples of a porous surgical buttress include those possessing a three dimensional structure, such as the textiles described in U.S. Pat. Nos. 7,021,086 and 6,443,964, the entire disclosures of each of which are incorporated by reference herein.

The porosity of the porous and/or swellable layer 40 and/or the substrate 30 may allow for the infiltration of biological fluids, molecules, including proteins, small molecules, RNA, etc., and/or cellular components which, in turn, may accelerate healing and/or the release kinetics of any therapeutic agent from the surgical buttress 24 of the present disclosure.

In aspects, materials used to form the layers of the surgical buttress may be biodegradable, so that the surgical buttress does not have to be retrieved from the body. The term “biodegradable” as used herein is defined to include both bioabsorbable and bioresorbable materials. By biodegradable, it is meant that the surgical buttress decomposes or loses structural integrity under body conditions (e.g., enzymatic degradation or hydrolysis), or is broken down (physically or chemically) under physiologic conditions in the body such that the degradation products are excretable or absorbable by the body.

Non-limiting examples of materials which may be biodegradable or non-degradable used in forming the various layers of the surgical buttress of the present disclosure include, but are not limited to, polyethylene glycol, poly(alkylene oxide) including poly(ethylene oxide) and poly(propylene oxide), poly(lactic acid), poly(glycolic acid), poly(alkylene carbonate) including poly(trimethylene carbonate), poly(dioxanone), poly(hydroxybutyrate), poly(phosphazine), poly(ethylene terephthalate), polyacrylamides, polyacrylate, polymethacrylate, poly(hydroxyethylmethylacrylate), poly(vinylpyrrolidone), poly(vinyl alcohols), polyacrylic acid, polyacetate, polycaprolactone, polypropylene, aliphatic polyesters, poly(glycerol ester) including poly(glycerol sebacate), poly(amino acids), copoly(ether-esters), polyalkylene oxalates, polyamides, poly(iminocarbonates), polyalkylene oxalates, polyoxaesters, polyorthoesters, polyurethane, poly(2-oxazoline), polymers with phosphoryl choline, sulfobetaine (SB), or carboxybetaine (CB) functional groups, and copolymers, block copolymers, homopolymers, blends and combinations thereof. The polymers may be in non-crosslinked or crosslinked form.

One embodiment of swellable materials is crosslinked poly(ethylene glycol) (PEG). PEG can be crosslinked by various crosslinking methods such as photochemical, free radical, click chemistry, NHS chemistry, addition chemistry such as Michael addition chemistry, thiol chemistry, Diels-Alder chemistry, condensation chemistry, genipin chemistry, urethane chemistry or supramolecular methods through hydrogen bonding or ionic or guest-host interactions. One embodiment of the crosslinking is by the NHS chemistry as illustrated in Example 1. The PEG before crosslinking can be linear with a molecular weight range of about 500 to 100,000 Dalton, or multi-arm with functionality of 3 to 10 and a molecular weight about 500 to 100,000 Dalton. Another embodiment is the crosslinked PEG degradable by introducing chemical bonds in the chain, which can break under physiological conditions such as hydrolysis. The degradation time can be tuned from 1 day to 1 year by changing the crosslinking density or the rate of hydrolysis of the ester bond for example. The swelling ratio can also be tuned from 1 to about 50 by changing the stoichiometric ratio, crosslinking density, the molecular weight between crosslinking points. The swelling ratio is defined as weight gain under physiological conditions vs the initial dry weight of the layer.

In aspects, natural biological polymers may be used in forming the various layers of the surgical buttress of the present disclosure. Suitable natural biological polymers include, but are not limited to, collagen, alginate, gelatin, hyaluronic acid, dextran, fibrin, fibrinogen, elastin, keratin, albumin, cellulose, oxidized cellulose, hydroxyethyl cellulose, hydroxypropyl cellulose, carboxyethyl cellulose, carboxymethyl cellulose, chitin, chitosan, and combinations thereof. In addition, natural biological polymers may be combined with any of the other polymeric materials described herein to produce a surgical buttress of the present disclosure.

The swellable layer optionally contains 0.05% to 7.5% antioxidants such as BHT to help with the stabilization during processing such as ETO and storage up to three years.

The swellable layer optionally contains 0.5% to 20% additives to improve the lubricity and the toughness of the material in the hydrated state. Examples of such additives include glycerol, low molecular weight PEG with molecular weight of 100 to 300.

The swellable and or porous layer may be formed by various crosslinking methods such as photochemical, free radical, click chemistry NHS chemistry, addition chemistry such as Michael addition chemistry, thiol chemistry, Diels-Alder chemistry, condensation chemistry, genipin chemistry, urethane chemistry or supramolecular methods through hydrogen bonding or ionic or guest-host interactions. The chemistry can be carried out in bulky or solution. The solvent can be organic such as methanol, ethanol or propanol. The preferred solvent is water or partially water. The working time prior to the gelation is between 5 minutes to 60 minutes. The stochiometric ratio of the reactants varies from 0.4 to 2.5. The concentration of the reactants varies from 1% to 100%. In the case of the aqueous reaction, the pH of the solutions varies from 5 to 10. The pH is controlled by a buffer such as phosphate buffer with concentration from 0.01 to 0.5M.

The porous structure of the swellable layer can be formed by various methods such as lyophilization process, salt out, selective leaching, electrospinning, etc.

The application of the layer to buttress can use a molding method in which the layer is formed in a tray and the buttress is positioned at the bottom or the top of the mold. The mold is made of metal and coated with non-sticking material such as PTFE. It can also be made of plastic material such as non-sticking PTFE or polyethylene etc. to facilitate the release of the swellable layer from the mold. The other application methods include spraying coating, slot die coating, electrospinning, hot pressing, etc.

In some aspects, the substrate 30 may be a non-woven substrate. In aspects, substrates include those formed of a biodegradable, porous matrix layer formed of a suitable material, for example a degradable polymer such as poly(lactic acid), poly(glycolic acid), poly(alkylene carbonate) including poly(trimethylene carbonate), polycaprolactone, poly(dioxanone), poly(hydroxybutyrate), poly(phosphazine). aliphatic polyesters, poly(glycerol ester) including poly(glycerol sebacate), poly(amino acids), copoly(ether-esters), polyalkylene oxalates, polyamides, poly(iminocarbonates), polyalkylene oxalates, polyoxaesters, polyorthoesters, and/or polyurethanes. The porous and/or swellable layer 40 may be formed of degradable polymers, polyethylene glycol, poly(alkylene oxide) including as poly(ethylene oxide) and poly(propylene oxide), alginate, collagen, gelatin, chitosan, dextran, hyaluronic acid, or a combination of these materials. In some aspects, the porous and/or swellable layer 40 may be a swellable material such as a hydrogel. The non-permeable layer 50 may be formed of degradable polymers, alginate, collagen, gelatin, chitosan, dextran, hyaluronic acid, thrombin, or a combination of these materials.

In aspects, the same material may be used to form both the porous and/or swellable layer and the non-permeable layer. This may be possible following methods within the purview of those skilled in the art, for example, cross-linking or physically pressing and compacting what would be a porous and/or swellable material to form a non-permeable material. The layer may be formed by various crosslinking methods such as photochemical, free radical, click chemistry NHS chemistry, addition chemistry such as Michael addition chemistry, thiol chemistry, Diels-Alder chemistry, condensation chemistry, genipin chemistry, urethane chemistry or supramolecular methods through hydrogen bonding or ionic or guest-host interactions. The chemistry can be carried out in bulky or solution. The solvent can be organic such as methanol, ethanol or propanol. The preferred solvent is water or partially water. The working time prior to the gelation is between 0.5 min to the 60 min. The stochiometric ratio of the reactants varies from 0.4 to 2.5. The concentration of the reactants varies from 1% to 100%. In the case of the aqueous reaction, the pH of the solutions varies from 5 to 10. The pH is controlled by a buffer such as phosphate buffer with concentration from 0.01 to 0.5M.

In aspects, the surgical buttress 24 may have a construction as depicted in FIG. 6, including the substrate 30, with the porous and/or swellable layer 40 formed over the substrate 30, and the non-permeable layer 50 formed over the porous and/or swellable layer 40. In use, the substrate 30 will be adjacent to the tissue.

In some aspects the surgical buttress may have a construction as depicted in FIG. 7, including the substrate 30, with the porous and/or swellable layer 40 formed over the substrate 30. In use, the substrate 30 will be adjacent to the tissue. The porous and/or swellable layer may on one side of the buttress or both sides. If the layer is on only one side of the buttress, it may be in contact with the tissue or away from the tissue.

In other aspects, the surgical buttress 24 may have a construction as depicted in FIG. 8, including the porous and/or swellable layer 40, the substrate 30 formed over the porous and/or swellable layer 40, and the non-permeable layer 50 formed over the substrate 30. In use, the porous and/or swellable layer 40 will be adjacent to the tissue.

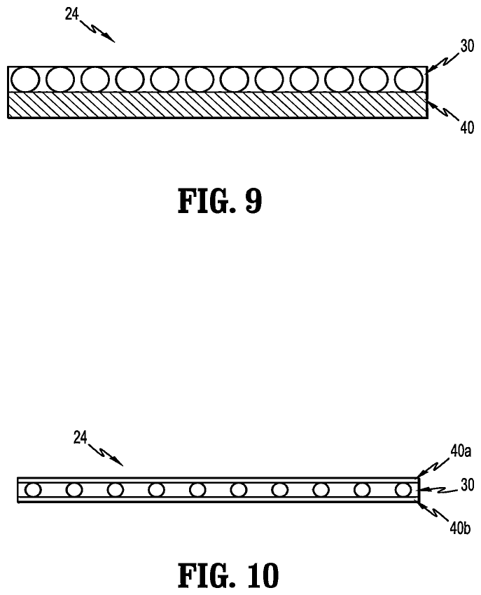

In some aspects the surgical buttress may have a construction as depicted in FIG. 9, including the porous and/or swellable layer 40, with the substrate 30 formed over the porous and/or swellable layer 40. In use, the porous and/or swellable layer 40 will be adjacent to the tissue.

In yet other aspects, the surgical buttress 24 may have a construction as depicted in FIG. 10, including the substrate 30 between two porous and/or swellable layers, 40a, 40b.

In aspects, the porous and/or swellable layer may penetrate into the pores of the substrate layer. In other aspects, the porous and/or swellable layer does not penetrate into the pores of the substrate layer.

While many of the depictions of the surgical buttress 24 show contiguous layers, it is to be understood that, in some embodiments, the layers may not be contiguous, i.e., the non-permeable layer 50 may extend beyond the borders of the substrate 30, the porous and/or swellable layer 40, or both. Similarly, the porous and/or swellable layer 40 may extend beyond the borders of the substrate 30. Although not depicted, layers of the surgical buttress 24 may also be non-contiguous, e.g., applied as a pattern to another layer. Any combination of the above may also be utilized. In yet other aspects (not shown), the porous and/or swellable layer may also be applied as a pattern on the substrate.

The substrate used to form the surgical buttress may have a pore volume from about 65% to about 90%, in aspects from about 70% to about 85%.

Substrates used to form surgical buttresses of the present disclosure may have a thickness from about 0.05 mm to about 0.5 mm, in embodiments from about 0.1 mm to about 0.2 mm.

In use, the porous and/or swellable layer 40 may swell when introduced to fluids and/or staple line exudate, but will be restricted by the staples, exerting additional compression on the staple line. Swelling of the porous and/or swellable layer 40 may increase compression exerted on the staple line by the substrate. Swelling of the porous and/or swellable layer 40 may also fill gaps which may exist between the buttress 24 and staples, the buttress 24 and the tissue, and/or the staples and the tissue.

The swelling ratio of the porous and/or swellable layer may vary from 1 to 50. The swelling ratio is defined as weight gain under physiological conditions vs. the initial dry weight of the layer. The pore size may vary from 0.5 microns to 250 microns. The porous and/or swellable layer 40 used to form the surgical buttress may have a pore volume from about 65% to about 99%, in aspects from about 70% to about 95%.

Porous and/or swellable layers used to form surgical buttresses of the present disclosure may have a non-compressed thickness from about 100 μm to about 2000 μm, in aspects from about 200 μm to about 1800 μm.

The non-permeable layer 50 acts as a physical barrier to leaks at the staple line and/or additional bleeding. The non-permeable layer 50 may also adsorb certain endogenous biological materials, including red blood cells, white blood cells, platelets, and the like, and thus aid in clot formation and/or wound healing.

Non-permeable layers used to form surgical buttresses of the present disclosure may have a thickness from about 5 μm to about 50 μm, in aspects from about 10 μm to about 30 μm.

Regardless of construction, in use the surgical buttress 24 will be compressible and will enable stapling through the surgical buttress and underlying tissue, without impeding function of the surgical stapler.

While the above description is directed to rectangular surgical buttresses, it is to be appreciated that any suitable configuration for a surgical buttress may be utilized in accordance with the present disclosure. For example, surgical buttresses having an clongate rectangular body with head and tail portions at the ends of the surgical buttress may be utilized. Any other suitable shape may be utilized, including circular or any other shape suitable use for surgical buttresses. For example, additional suitable surgical buttresses include those disclosed in U.S. Pat. Nos. 10,682,140, 8,157,151, 8,561,873 and 9,693,772, the entire disclosures of each of which are incorporated by reference herein.

Therapeutic agents may be added to a surgical buttress of the present disclosure for delivery to a patient. Suitable therapeutic agents include, but are not limited to, drugs, small molecules, amino acids, peptides, polypeptides, proteins, polysaccharides, muteins, immunoglobulins, antibodies, cytokines (e.g., lymphokines, monokines, chemokines), blood clotting factors, hemopoietic factors, interleukins (1 through 18), interferons (β-IFN, α-IFN and γ-IFN), erythropoietin, nucleases, tumor necrosis factor, colony stimulating factors (e.g., GCSF, GM-CSF, MCSF), insulin, anti-tumor agents and tumor suppressors, blood proteins, fibrin, thrombin, fibrinogen, synthetic thrombin, synthetic fibrin, synthetic fibrinogen, gonadotropins (e.g., FSH, LH, CG, etc.), ellagic acid, tranexamic acid, hormones and hormone analogs (e.g., growth hormone, luteinizing hormone releasing factor), vaccines (e.g., tumoral, bacterial and viral antigens), somatostatin, antigens, blood coagulation factors, growth factors (e.g., nerve growth factor, insulin-like growth factor), bone morphogenic proteins, TGF-B, protein inhibitors, protein antagonists, protein agonists, nucleic acids, such as antisense molecules, DNA, RNA, RNAi, oligonucleotides, polynucleotides, cells, viruses, anti-inflammatory agents, anti-bacterial agents, antimicrobial agents, antifungal agents, and ribozymes. In aspects, the therapeutic agent applied to a surgical buttress of the present disclosure may include an anti-tumor agent and/or tumor suppressor, referred to, in aspects, as a “chemotherapeutic agent” and/or an “antineoplastic agent.” Suitable chemotherapeutic agents include, for example, paclitaxel and derivatives thereof, docetaxel and derivatives thereof, abraxane, tamoxifen, cyclophosphamide, actinomycin, bleomycin, dactinomycin, daunorubicin, doxorubicin, doxorubicin hydrochloride, epirubicin, mitomycin, methotrexate, fluorouracil, gemcitabine, gemcitabine hydrochloride, carboplatin, carmustine (BCNU), methyl-CCNU, cisplatin, ctoposide, camptothecin and derivatives thereof, phenesterine, vinblastine, vincristine, goserelin, leuprolide, interferon alfa, retinoic acid (ATRA), nitrogen mustard alkylating agents, piposulfan, vinorelbine, irinotecan, irinotecan hydrochloride, vinblastine, pemetrexed, sorafenib tosylate, everolimus, erlotinib hydrochloride, sunitinib malate, capecitabine oxaliplatin, leucovorin calcium, bevacizumab, cetuximab, ramucirumab, trastuzumab, atczolizumab, canakinumab, combinations thereof, and the like. Other suitable therapeutic agents include immunotherapeutics, including pembrolizumab, nivolumab, atezolizumab, durvalumab, cemiplimab, and/or Osimertinib, cell targeting therapies, including iplimunmab, combinations thereof, and the like.

In aspects, paclitaxel and/or paclitaxel derivatives may be used as the therapeutic agent. Paclitaxel may have various forms, referred to herein as “polymorphs,” including amorphous paclitaxel, crystalline paclitaxel, sometimes referred to as crystalline paclitaxel dihydrate, and/or anhydrous paclitaxel, or mixtures thereof.

Any therapeutic agent may be added to any layer of the surgical buttress 24, including the substrate 30, the porous and/or swellable layer 40, the non-permeable layer 50, or any combination thereof. For example, the therapeutic agent may be combined with the materials used to form the various layers, so that it is incorporated within the material forming the layer(s), or for non-woven or porous and/or swellable materials, the therapeutic agent may be present within the pores of the non-woven or porous and/or swellable material.

In aspects, as noted above, a surgical buttress of the present disclosure is provided to reinforce and seal the lines of staples applied to tissue by a surgical stapling apparatus. Upon application to a site of bleeding tissue, the surgical buttress may affect hemostasis of said tissue. As used herein, the term “hemostasis” means the arrest of bleeding.

In addition to providing hemostasis at the site of application of the surgical buttress, the surgical buttresses of the present disclosure may also provide for treatment of tissue with a therapeutic agent at both the site of implantation and elsewhere in the body.

In accordance with the present disclosure, buttresses having desirable characteristics may be designed and/or constructed considering at least some of the following factors:
Buttress and properties of the various layers, including the porous and/or swellable layer and the non-permeable layer, result in increasing leak pressures which may also improve staple line hemostasis.
Certain materials for the substrate, for example polyglycolic acid, which is stiffer, may provide more mechanical compression in the staple line than other materials; buttress stiffness may best be altered by tuning density and thickness of the substrate.
Increasing density and/or thickness of the substrate beyond a certain point may adversely impact staple formation, firing, and retraction forces, and staple line abrasiveness.
Porous and/or swellable layers that swell with fluid absorption may enable greater final buttress thicknesses.
Porous and/or swellable layers that produce expansile forces during swelling may tend to increase buttress compression on tissue and around staple hole defects; larger expansile forces may be desirable to improve resistance to leaks and bleeding both intra-operatively and post-operatively.
Elastic properties of the buttress and/or the porous and/or swellable layer allow expansion lateral to the buttress edges, enabling coverage of cut edge and tissue prone to tearing adjacent the staple line.
Increasing expansile forces beyond a certain point may adversely impact staple formation and firing and/or retraction forces.
Degree of swelling and expansile forces can be readily tuned and controlled using some synthetic materials, for example polyethylene glycol, when compared with natural materials such as collagen and alginate.
Greater swelling may increase endogenous growth factor absorption, leading to improved healing and fewer post-operative leaks.
Blood clots in the layers of the buttress (either passively or with the aid of active clotting agents)
Platelets in clot supply growth factors to healing staple line.
Buttress mechanically protects clot from delaminating from staple line.
Greater clot adhesivity may reduce likelihood of leaks and bleeding and may enable a larger reservoir of growth factors for staple line healing.
New tissue grows into buttress over time.
Buttress may protect staple line from the formation of adhesions post-operatively

### EXAMPLE 1

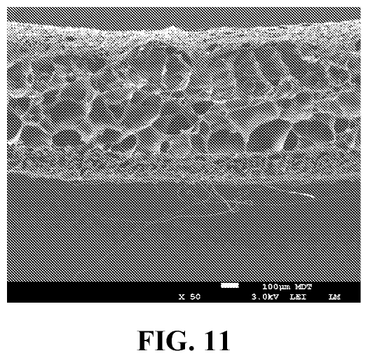

Preparation the buttress described herein. 4.5 mL of deionized water (pH ca 6.7) was added to a 20 mL glass vial with 0.3 grams of poly(ethylene glycol) diamine having a molecular weight 10,000 Dalton and the mixture was vortexed to make a homogeneous solution. A second solution of 0.15 grams of 4-arm poly(ethyelene glycol) succinimidyl glutarate ((4-arm PEG SG)) with a molecular weight of 10,000 Dalton in 1.5 mL deionized water was made similarly. The two solutions were mixed and vortexed to make a homogeneous solution to start the crosslinking reaction by NHS chemistry. A stainless 10×4 cm tray with a depth of 1 mm and coated with PTFE was filled with the solution prior to the gelation in about 10 minutes. Immediately a poly(glycolic acid) non-woven buttress sheet with 0.15 mm thickness was laid on the top of the tray. The tray sat on a flat surface for 2 hours before being put in a −80° C. freezer for 1 hour. The tray was transferred to a lyophilizer. The lyophilization was at −20° C. in an oven under high vacuum for 24 hours and the remaining residual water was removed to a room temperature oven for another 24 hours under high vacuum. The coated buttress was removed by peeling of the tray. The cross-section of the coated buttress was imaged by scanning electron microscopy (SEM), which showed good integration of the porous crosslinked PEG layer to the buttress. The SEM image of the coated buttress is provided as FIG. 11.

### EXAMPLE 2

In a primary, acute, in vivo preclinical setting, the buttress described herein, when used with a surgical stapler, demonstrated significantly fewer staple line bleeding events, and significantly higher stapler line leak pressure, when compared to other commercially available buttress materials:
Comparative Example A: Tri-Staple™ 2.0 Reinforced Reload (Medtronic)
Comparative Example B: GORE® SEAMGUARD® (GORE)
Comparative Example C: ECHELON ENDOPATH™ Staple line Reinforcement (Ethicon)

Buttressed staple lines were placed longitudinally along the anti-mesenteric border of canine small bowel maintaining at least 50% patency of the lumen. Upon release of the tissue from the surgical stapler, the occurrence of a bleeding event was recorded (defined as any visible oozing of blood from the staple line after initial blotting), and the bleeding severity was assessed and recorded based on the following Likert Scale:

TABLE 1
Hemostasis rating scale¹

| Score | Description |
|---|---|
| 0 | No bleeding at the tissue site after initial |
| | blotting of staple line |
| 1 | Blood oozing at the tissue site; stops prior to |
| | 1 min; no intervention needed |
| 2 | Blood oozing, still progressive after 1 min |
| | through 3 min; no intervention needed |
| 3 | Blood oozing at tissue site after 3 min, mild |
| | intervention needed (i.e. cautery) |
| 4 | Significant bleeding requiring intervention such |
| | as extensive coagulation of ligation with clips |

¹Siegel JM, et al. Journal of Advances in Medical and Pharmaceutical Sciences, 1(1): 30-39, 2014.

Leak testing was performed for each buttressed staple line using a Z-axis system (Isaac HD Muti-Function Leak Tester). The section of bowel that contained the staple line was clamped off for testing, and the staple line was wetted with saline. The Z-axis needle was inserted into the pouch longitudinally and pressure was increased incrementally until a leak (defined as the appearance of air bubbles at the staple or cut line) was observed and that pressure was recorded.

### Results

### Bleeding

The buttress described herein resulted in no staple line bleeding for all samples tested, and significantly fewer bleeding events than Comparative Example C (Tables 3 and 4).

TABLE 2
Bleeding Severity (based on Scale)

| | | | Mean ± Standard |
|---|---|---|---|
| Group | N | Median | Deviation |
| Buttress described herein | 7 | 0 | 0.0 ± 0.0 |
| A (Medtronic) | 7 | 0 | 0.7 ± 1.0 |
| B (GORE) | 7 | 0 | 0.0 ± 0.0 |
| C (Ethicon) | 6 | 2 | 1.7 ± 0.8 |

TABLE 3
Bleeding Events

| Group | % | Events | P-Value* |
|---|---|---|---|
| Buttress described herein | 0.0 | 0/7 | — |
| A (Medtronic) | 42.9 | 4/7 | 0.192 |
| B (GORE) | 0.0 | 0/7 | 1.000 |
| C (Ethicon) | 83.3 | 5/6 | 0.005 |

*P-value based on comparison of the buttress described herein and each other group individually using a two-proportion, Fisher exact test. A p-value < 0.050 is considered statistically significant.

### Leak Testing

The buttress described herein resulted in significantly higher staple line leak pressure as compared to all other Comparative Examples, as determined by One-way ANOVA with Tukey Pairwise Comparisons (Tables 4 and 5). A p-value<0.050 is considered statistically significant. No other differences between Examples were observed.

TABLE 4
Staple Line Leak Pressure Summary

| | | Mean ± Standard |
|---|---|---|
| Group | N | Deviation (mm Hg) |
| Buttress described herein | 7 | 173.1 ± 35.2 |
| A (Medtronic) | 6 | 133.7 ± 30.5 |
| B (GORE) | 7 | 106.2 ± 16.2 |
| C (Ethicon) | 6 | 94.1 ± 18.7 |

TABLE 5
Tukey Simultaneous Tests for Differences of Means

| Difference of | Difference | SE of | | T- | Adjusted |
|---|---|---|---|---|---|
| Levels | of Means | Difference | 95% CI | Value | P-Value |
| Buttress | 40.4 | 14.5 | (0.2, 80.6) | 2.79 | 0.048 |
| described | | | | | |
| herein-A | | | | | |
| B-A | −27.5 | 13.9 | (−66.1, 11.1) | −1.98 | 0.227 |
| C-A | −39.6 | 14.5 | (−79.8, 0.6) | −2.74 | 0.054 |
| B-buttress | −67.9 | 14.5 | (−108.1, | −4.69 | 0.001 |
| described | | | −27.7) | | |
| herein | | | | | |
| C-buttress | −80.0 | 15.0 | (−121.8, | −5.33 | 0.000 |
| described | | | −38.3) | | |
| herein | | | | | |
| C-B | −12.1 | 14.5 | (−52.3, 28.1) | −0.84 | 0.836 |

Individual confidence level = 98.91%

### Chronic Preclinical Model

Furthermore, using the canine preclinical model and leak test method described previously, the buttress described herein resulted in significantly higher staple line leak pressure in a chronic assessment when compared to Comparative Example A, as determined by One-way ANOVA with Tukey Pairwise Comparisons. Staple line leak pressures were determined post-mortem on post-operative day 3 (Tables 6 and 7).

TABLE 6
Post-Mortem Staple Line Leak Pressure
Summary, Post-Operative Day 3

| | | Mean ± Standard |
|---|---|---|
| Group | N | Deviation (mm Hg) |
| Non-buttressed | 6 | 81.1 ± 19.9 |
| A (Medtronic) | 6 | 71.9 ± 14.2 |
| Buttress described | 6 | 110.1 ± 35.5 |
| herein | | |

TABLE 7
Tukey Simultaneous Tests for Differences of Means

| Difference of | Difference | SE of | | T- | Adjusted |
|---|---|---|---|---|---|
| Levels | of Means | Difference | 95% CI | Value | P-Value |
| A-Non-buttressed | −9.3 | 14.4 | (−46.6, | −0.64 | 0.798 |
| | | | 28.0) | | |
| Buttress described | 28.9 | 14.4 | (−8.4, | 2.01 | 0.143 |
| herein-Non- | | | 66.2) | | |
| buttressed | | | | | |
| Buttress described | 38.2 | 14.4 | (0.9, | 2.66 | 0.045 |
| herein-A | | | 75.5) | | |

Individual confidence level = 97.97%

It will be understood that various modifications may be made to the aspects disclosed herein. Therefore, the above description should not be construed as limiting, but merely as an exemplification of preferred aspects. Those skilled in the art will envision other modifications within the scope and spirit of the present disclosure. Such modifications and variations are intended to come within the scope of the following claims.

1-20. (canceled)

21. A surgical stapling device, comprising:
an end effector including an anvil jaw member and a staple cartridge jaw member pivotally coupled to one another, the anvil jaw member and the staple cartridge jaw member being relatively movable such that the end effector is movable between an open position and a clamped position; and
a surgical buttress attached to the anvil jaw member, the staple cartridge jaw member, or both, the surgical buttress including:
a substrate having a first thickness; and
a swellable layer having a second thickness, wherein a ratio of the second thickness to the first thickness is between 2 to 4.

22. The surgical stapling device of claim 21, wherein the swellable layer has a swelling ratio of at least 1 and up to 50, wherein the swelling ratio is weight gain under physiological conditions as compared to an initial dry weight of the swellable layer.

23. The surgical stapling device of claim 21, wherein the swellable layer comprises crosslinked polyethylene glycol (PEG).

24. The surgical stapling device of claim 23, wherein the crosslinked PEG is degradable by hydrolysis.

25. The surgical stapling device of claim 21, wherein the swellable layer has a pore size from 0.5 microns to 250 microns.

26. The surgical stapling device of claim 21, wherein the surgical buttress further comprises a non-permeable layer having a third thickness.

27. The surgical stapling device of claim 26, wherein the third thickness is less than one-third of the second thickness.

28. The surgical stapling device of claim 21, wherein the substrate has pores in an amount from 65% to 90% of a volume of the substrate.

29. The surgical stapling device of claim 21, wherein the swellable layer has pores in an amount from 65% to 99% of a volume of the swellable layer.2

30. A surgical stapling device for stapling tissue, the surgical stapling device comprising:
an end effector having an anvil jaw member and a staple cartridge jaw member pivotally coupled to one another, the anvil jaw member and the staple cartridge jaw member being configured to move relative to each other between an open configuration and a closed configuration; and
a surgical buttress attached to the anvil jaw member, the staple cartridge jaw member, or both, the surgical buttress comprising:
a first, non-swellable layer having a first thickness; and
a second, swellable layer of swellable material having a second thickness, the second layer attached to the first layer, wherein a ratio of the second thickness to the first thickness is between 2 to 4.

31. The surgical stapling device of claim 30, further comprising a non-permeable layer having a third thickness that is less than each of the first thickness and the second thickness.

32. The surgical stapling device of claim 30, wherein the second layer comprises polyethylene glycol (PEG).

33. The surgical stapling device of claim 32, wherein the polyethylene glycol is crosslinked via N-hydroxysuccinimide (NHS) chemistry.

34. The surgical stapling device of claim 32, wherein the first layer comprises a polyglycolic acid.

35. The surgical stapling device of claim 30, wherein the swellable material has a swelling ratio of at least 1 and up to 50, wherein the swelling ratio is weight gain under physiological conditions as compared to an initial dry weight of the second layer.

36. The surgical stapling device of claim 30, wherein the first layer has pores in an amount from 65% to 90% of a volume of the first layer.

37. The surgical stapling device of claim 30, wherein the second layer has pores in an amount from 65% to 99% of a volume of the second layer.

38. A surgical buttress for use in surgical stapling, the surgical buttress comprising:
a first layer, wherein the first layer comprises polyglycolic acid and has a first thickness;
a second, swellable layer attached to a tissue-facing side of the first layer, wherein the second layer:
comprises polyethylene glycol (PEG);
has a second thickness, wherein a ratio of the second thickness to the first thickness is between 2 to 4; and
has a swelling ratio of at least 1 and up to 50, wherein the swelling ratio is weight gain under physiological conditions as compared to an initial dry weight of the second layer.

39. The surgical buttress of claim 38, wherein the second layer has a non-compressed thickness of 200 μm to about 1800 μm.

40. The surgical buttress of claim 38, wherein the second layer is a coating on the first layer.
